(19) United States
(12) Patent Application Publication
(10) Pub. No.: US 2005/0008710 A1
(43) Pub. Date: Jan. 13, 2005

(54) Method for screening for endothelin-receptor antagonist activity and for treating conditions caused by endothelin

(75) Inventor: Ven Subbiah, Greenville, NC (US)

Correspondence Address: DANIELS DANIELS & VERDONIK, P.A., SUITE 200 GENERATION PLAZA, 1822 N.C. HIGHWAY 54 EAST, DURHAM, NC, 27713, US

(73) Assignee: PhytoMyco Research Corporation, Greenville, NC (US)

(21) Appl. No.: 10/885,353

(22) Filed: Jul. 6, 2004

Related U.S. Application Data
(60) Provisional application No. 60/485,777, filed on Jul. 9, 2003.

(51) Int. Cl. C12Q 1/37; A61K 35/78

(52) U.S. Cl. 424/725; 435/23; 424/769; 424/770; 424/754

(57) ABSTRACT

Aliquots of extracts from ethnopharmacological plants that have activity against the effects of sarafotoxins present in snake venom are isolated and identified as antagonists of endothelin using a fluorescence-based assay. A process is provided for the identification of an antagonist of an endothelin selected from the group consisting of endothelin-1, endothelin-2, endothelin-3 and mixtures thereof. The process comprises extraction of ethnopharmacological plants with a solvent followed by evaporation of the solvent to form an aliquot containing at least one component of the extract, optionally purifying and isolating one or more component by chromatography, and subjecting the aliquot or purified component to a competitive fluorescent binding assay using biotinylated endothelin-1, wherein the plants having activity against the effects of one or more sarafotoxins present in snake venom.

### CROSS REFERENCE TO RELATED APPLICATIONS

This application is related to and claims priority to Provisional Application Ser. No. 60/485,777, filed Jul. 9, 2003, the disclosure of which is incorporated in its entirety by reference herein.

### FIELD OF THE INVENTION

The present invention relates to methods for screening a mixture of components of an extract of a plant or other natural source using a fluorescent-based ligand-receptor interaction assay technique to identify a compound or a mixture of compounds that exhibits endothelin antagonist activity. The invention further relates to treatment of hypertension, pulmonary hypertension, renal failure and/or kidney failure with such compound or mixture of compounds.

### BACKGROUND OF THE INVENTION

Endothelin is a cyclic peptide that causes a strong, long-lasting vasoconstriction effect in humans, and is largely responsible for or involved in disease states such as hypertension, asthma, acute renal failure, cardiac infarction, cerebral apoplexy, angina pectoris, and cerebral vasospasm. As a result, there is a strong medical need to find, identify, and provide antagonists of endothelin. There is also a need to provide methods that are simple and useful to identify endothelin antagonist drug substances. Such antagonists can inhibit the effect of endothelin and are expected to be useful for the treatment and prevention of these disease states. In another aspect, identification of agonist molecules that exhibit endothelin receptor binding and responses analogous to that of endothelin can be useful to aid in identification of binding sites in receptors for endothelin. This binding information can also be useful for the discovery of antagonists of endothelin-1, and/or of endothelin-2, and/or of endothelin-3.

A drug or a biologically active molecule can produce a change in a human or animal when it interacts with an endogenous macromolecular receptor molecule such as an endothelin receptor. An endothelin agonist is a drug that increases a physiological activity and/or biochemical function of the endothelin receptor when the drug is bound to or forms a complex with the receptor. An endothelin antagonist is a drug or biologically active molecule that reduces or removes or partially or totally inhibits a physiological activity and/or biochemical function of the receptor when the antagonist is bound to or forms a complex with the endothelin receptor. The biological function or process in which the receptor participates can have an excitatory or an inhibitory mechanism.

Each endothelin agonist and each endothelin antagonist drug exhibits a relative affinity for the endothelin receptor molecule to which it binds to form a drug-receptor complex. The relative affinity of an agonist drug or of an antagonist drug can be represented by an equilibrium association constant or equilibrium dissociation constant for the drug-receptor complex. An agonist and an antagonist can bind to substantially similar sites on a receptor molecule or to different sites on a receptor. The binding affinity between a drug and a receptor molecule can depend on a number of parameters including the molecular structure of the drug, the number of rotational degrees of freedom of the drug molecule, the presence of charged or polarized functional groups in the drug molecule, the present of functional groups having the ability to form hydrogen bonds with the receptor molecule and with other molecules proximal to the receptor binding site, the conformational flexibility of the drug molecule, the ability of a drug to form van der Waals interactions with functional groups in a receptor which can be the result of favorable molecular orbital overlap between drug and receptor molecular orbitals of similar energy that leads to a net lowering of energy levels and increased binding, the kind and number of substituents in a drug molecule, and the relative spacial or stereospecific orientation of the functional groups in the drug that interact with functional groups in the receptor molecule. In a group of molecules that exhibit antagonist activity for an endothelin receptor, different antagonist molecules can bind to substantially the same site on the receptor molecule as other antagonists or to different sites on the receptor molecule, or to both the same and different sites on the endothelin receptor. An antagonist may remove either an excitatory or inhibitory mechanism controlling bodily processes.

In vitro screening assays have been used to identify active compounds that exhibit agonist or antagonist activity as ligands that bind to a receptor such as a protein, antibody, or enzyme that can be present at the surface or inside of a cell or that can be isolated from a cell or tissue or blood of an animal or that can be prepared by recombinant methods. Cells containing receptors used in screening assays can sometimes be grown in culture media or isolated from living or dead tissue, and a receptor useful in an assay can be isolated from such cells. In a receptor-ligand binding assay, active compounds that are found to exhibit a desired level of binding affinity to a receptor can be hit compounds or lead compounds.

By “hit compounds” is meant the compounds exhibiting binding affinity and are ET_{A}-receptor selective, ET_{B} selective or non selective for ET_{A} and ET_{B}. By “lead compounds” is meant the compound exhibiting binding affinity to ET_{A} receptor selective. Such compounds can be useful as pharmaceutical or therapeutic agents or as leads in the discovery of pharmaceutical or therapeutic agents. For an antagonist identified by a screening process, a dose-response relationship between the antagonist and a receptor can be determined relative to the dose-response of a standard or reference antagonist compound. Thereafter, structural analogs of the antagonist can be prepared and the dose-response relationships of these compounds can be used to define the pharmacophore or region of molecular stereospecific binding activity between the antagonist and the receptor.

In a drug discovery process, random screening assays that subject an aliquot of a sample containing naturally occurring or synthetic compounds or a mixture of such compounds to a screening procedure have been used to identify the presence of one or more active compounds in the aliquot. Active compounds are considered to be those compounds that exhibit a predetermined or better than predetermined level of activity in an in vitro biological assay system. To increase the probability of finding an active antagonist compound from a screening assay, a large number of compounds or mixtures of compounds are often examined. These compounds are often diverse in structure and origin and can be expensive and time-consuming to screen and analyze. It is therefore desirable to be able to reduce the number of compounds examined and shorten the time required to find compounds that exhibit antagonist activity against a receptor.

The source of a compound or a mixture of compounds typically subjected to a screening assay is frequently a library of compounds that have been prepared by synthetic chemical processes and/or from randomly selected natural product sources such as isolates from plant and animal sources. It is frequently desired that a library of compounds used for random screening be as diverse as possible and contain compounds from a variety of different sources to optimize the changes of finding an active compound in a screening assay.

In certain screening assays, radioisotopic labeling of reference compounds used as reagents in competitive binding assays has been employed. However, radionuclide-labeled compounds for these assays can be expensive, unstable, and time consuming to prepare, and the specific radioactivity decays with time as a function of the isotope used. Radionuclides always require isolation of the work environment where an assay is performed, and often require dedication of apparatus for their preparation, handling, and analysis. Monitoring of human exposure and health risks is required; waste disposal and transportation is regulated and can be complicated; and substantial technical training and documentation is mandatory. In other screening assays, fluorescent-labeled antibodies have been used in immunoassays. However, these too can be expensive, and can be sensitive to denaturing during handling. Thus, a screening procedure that obviates the need for radionuclide labeling and handling or eliminates the need for antibodies will be useful.

### BRIEF SUMMARY OF THE INVENTION

The present invention provides a method of detecting the presence of an antagonist of endothelin in an aliquot of an extract of an ethnopharmacological plant, the method comprising
(a) admixing said aliquot and a volume of a solution containing a control amount of biotinylated endothelin-1 into a diagnostically effective plurality of receptors for endothelin-1, said receptors fixed to a plate or substrate;
(b) incubating the admixture in (a) for a time and at a temperature sufficient to allow competitive binding of said antagonist and said biotinylated endothelin-1 to said fixed receptors to occur to form a combination of receptor-bound-antagonist and receptor-bound-biotinylated endothelin-1 as bound receptors;
(c) washing said bound receptors in (b) with a fluorescent-dye-free buffer solution to remove unbound biotinylated endothelin-1;
(d) adding an excess (e.g., twofold or fourfold concentration) of a solution of streptavidin-Alexa-568 fluorescent dye conjugate to the washed bound receptors in (c);
(e) incubating the streptavidin-Alexa-568 fluorescent dye conjugate with the washed bound receptors of step (c) for a time and at a temperature sufficient to allow binding between biotin and streptavidin to occur to form fluorescent-dye-streptavidin-biotin-endothelin-1-bound receptors in the presence of non-Alexa containing antagonist-bound-receptors;
(f) washing the receptors in (e) with a fluorescent-dye-free buffer solution; and
(g) determining the level of fluorescence emitted from the washed receptors in (f) relative to the level of a fluorescent control,
   wherein said control comprises a comparable plurality of receptors treated as in (a) to (f) but in the absence of the aliquot of said extract, and
   wherein said extract has an activity against the effects of one or more sarafotoxin present in snake venom.

The current invention also provides compositions and compounds that exhibit endothelin antagonist activity and methods to identify such compounds. In one aspect, the present invention provides a fluorescence-based screening assay that can be used to identify endothelin antagonist compounds. Each such identified antagonist exhibits an affinity for binding to an endothelin receptor that is measured relative to the binding affinity of a reference antagonist compound. The assay can be useful to identify and optimize the structure of pharmacological agents having endothelin antagonist activity. These agents can be useful in the diagnosis and treatment of disease.

In particular, the present invention provides a process for the identification of an antagonist of an endothelin selected from the group consisting of endothelin-1, endothelin-2, endothelin-3 and mixtures thereof, said process comprising extraction of ethnopharmacological plants with a solvent followed by evaporation of the solvent to form an aliquot containing at least one component of the extract, optionally purifying said component by chromatographic means, and subjecting said aliquot or said component to a competitive fluorescent binding assay comprising biotinylated endothelin-1, wherein said plants having activity against the effects of one or more sarafotoxin present in snake venom.

More specifically, in accordance with the invention it has been discovered that endothelin and a snake venom component known as sarafotoxin are similar. Certain plants are useful to obtain extracts which block the effects of sarafotoxin. Endothelins show a high degree of homology with the sarafotoxins extracted from snake venom, and shares several pharmacological properties such as inductions of disturbances in ECG and also competes with the endothelin-receptor finding sites in heart and brain tissues. The sequence homology of endothelins (“ET”) with sarafotoxins suggests a common evolutionary.

The use of plants to treat snake bites has long been recognized and documented. Thus, the relationship between ET and sarafotoxins from snake venom has lead to the invention wherein plants effective against such sarafotoxins are investigated to determine effectiveness against ET. Such plants have been extensively identified and catalogued in databases which are readily available. The natural product databases cataloging such plants include and are not limited to the NAPRALERT database and the Chapman Hall natural product database. The plants identified have been systematically extracted and fractionated on solid phase extraction (SPE) chromatography to enhance the detecting capability.

Thus, in another aspect, the present invention provides a fluorescence-based screening assay that can be used to identify endothelin antagonist compounds in extracts and concentrates from ethnopharmacological plants, which plants are otherwise used in the preparation of potions, sauves, ointments, and the like to treat by oral or topical or implant means of administration the effects of sarafotoxins present in snake venom, particularly, in viper venom. Such sarafotoxins, like ET, can exhibit pharmacological properties such as induction of disturbances in heart rhythm that are identified as abnormal patterns in electrocardiographs (ECGs). Sarafotoxins also have an affinity for certain endothelin-receptor binding sites in heart and brain tissues. Both endothelin and sarafotoxins can exhibit potent inotropic and negative chronotropic effects on isolated perfused hearts and each can induce coronary vasospasm, severe arrhythmia, atrioventricular block and lethal ventricular fibrillation. Competitive endothelin antagonists of this invention can mediate or prevent or reduce the effects of endothelin in a human patent and act to prevent or limit the frequency and intensity in a human of one or more clinically adverse event selected from the group consisting of coronary vasospasm, severe arrhythmia, atrioventricular block, lethal ventricular fibrillation, and combinations thereof, for example to levels less than 90%, preferably to levels less than 75%, more preferably to levels less than 50%, more preferably to levels less than 10%, and most preferably to levels less than 5% of the frequency and/or intensity of such event that can occur in a human in the absence of said antagonist. Sarafotoxin S6c, also known as SRTX-c, has the structure Cys-Thr-Cys-Asn-Asp-Met-Thr-Asp-Glu-Glu-Cys-Leu-Asn-Phe-Cys-His-Gln-Asp-Val-Ile-Trp with disulfide Bridges at positions 1-15 and 3-11, and is an ET_{B} agonist that exhibits strong vasoconstrictor activity. Atractaspis engaddensis sequence Sarafotoxin S6b, also known as SRTX-b, has the structure Cys-Ser-Cys-Lys-Asp-Met-Thr-Asp-Lys-Glu-Cys-Leu-Tyr-Phe-Cys-His-Gln-Asp-Val-Ile-Trp with disulfide bridges at positions 1-15 and 3-11, and is an ETA agonist that increases intracellular Ca²⁺.

In one embodiment, a preferred ethnopharmacological plant is a plant useful in the treatment of the effects of sarafotoxins as may be identified from the aforementioned databases and the like. The plant or part of the plant, preferably in a ground or powdered form, is extracted with an organic solvent such as ether or dichloromethane to provide a natural crude extract. This extract is then subjected to high-pressure liquid chromatography to separate the component ingredients from each other or to separate groups of at least two component ingredients from other ingredients and/or from other groups of ingredients. The activity or each of the separated or fractionated components and/or mixtures of components of the extract of the ethnopharmacological plant is then measured in a fluorescence-based assay. Isolated extracts of a preferred ethnopharmacological plant are active when taken orally in the treatment of the effects of sarafotoxins, and because the extracts can be ingested by humans, the toxicity of the orally administered extract is generally pharmaceutically acceptable. This provides an advantage in the screening process because at the time the drug development process begins, toxicity and activity information is available to guide laboratory preparations of crude extracts/semi-purified compounds, to devise dosing regiments in test animals, and in certain cases, to develop formulations of drugs for pre-clinical bioassays when active principles are identified.

Endothelins are a family of potent vasoconstrictor peptides comprising three isoforms. It is found in a host of mammalian species and is thought to have a potent effect in both the cardiovascular and central nervous systems. Endothelin-1 (ET-1), Endothelin-2 (ET-2) and Endothelin-3 (ET-3) have 21 amino acids and two disulfide bridges. Endothelin is found to circulate in the blood in significant levels and is thought to be involved with increasing blood pressure upon receptor binding.

(19) Endothelins act on two pharmacologically distinct subtypes of G protein-coupled receptors, endothelin-A and endothelin-B that are expressed on a wide variety of vascular and non-vascular cells. Human endothelin-1 is a peptide that consists of 21 amino acid residues, and has very strong vascular smooth muscle constrictor activity and vasopressor activity. Big endothelin-1, a precursor of endothelin-1, is comprised of about 40 amino acid residues and exists as an intermediate in the course of its biosynthesis. Human endothelin-1, also referred to as endothelin-alpha, has the amino acid sequence represented as: Cys Ser Cys Ser Ser Leu Met Asp Lys Glu Cys Val Tyr Phe Cys His Leu Asp Ile Ile Trp. Endothelin I human, porcine is available from Sigma as ET-1, catalog number 45187 with amino acid sequence of Cys-Ser-Cys-Ser-Ser-Leu-Met-Asp-Lys-Glu-Cys-Val-Tyr-Phe-Cys-His-Leu-Asp-Ile-Ile-Trp [Disulfide Bridges: 1-15; 3-11]. Human endothelin-2 has the amino acid sequence represented as: Cys Ser Cys Ser Ser Trp Leu Asp Lys Glu Cys Val Tyr Phe Cys His Leu Asp Ile Ile Trp. Endothelin-2 is available from Sigma as ET-2, catalog number E9012, with amino acid sequence of Cys-Ser-Cys-Ser-Ser-Trp-Leu-Asp-Lys-Glu-Cys-Val-Tyr-Phe-Cys-His-Leu-Asp-Ile-Ile-Trp [Disulfide Bridges: 1-15; 3-11]. Human endothelin-3, also referred as endothelin-gamma has the amino acid sequence represented as: Cys Thr Cys Phe Thr Tyr Lys Asp Lys Glu Cys Val Tyr Tyr Cys His Leu Asp Ile Ile Trp. Endothelin 3 human, rat is available from Sigma as ET-3, catalog number 45189 with amino acid sequence of Cys-Thr-Cys-Phe-Thr-Tyr-Lys-Asp-Lys-Glu-Cys-Val-Tyr-Tyr-Cys-His-Leu-Asp-Ile-Ile-Trp [Disulfide Bridges: 1-15; 3-11]. Endothelin-3 differs from endothelin-1 and endothelin-2 in 6 residues of 21 amino acid residues. Endothelin-3 is weaker in vascular smooth muscle constrictor activity and vasopressor activity than endothelin-1 and endothelin-2. Endothelin-3 forms a receptor system different from those of endothelin-1 and endothelin-2. Endothelin-1 is the most potent vasoconstrictor substance known. Endothelin-1, endothelin-2, and endothelin-3 are produced from corresponding longer intermediates termed big endothelin-1, big endothelin-2, and big endothelin-3 (38-41 amino acids) by mammalian cell, including epithelial, interstitial and neuronal cells, and have distinct but partially overlapping tissue distributions. Big Endothelin 38 human is available from Sigma with the structure of Cys-Ser-Cys-Ser-Ser-Leu-Met-Asp-Lys-Glu-Cys-Val-Tyr-Phe-Cys-His-Leu-Asp-Ile-Ile-Trp-Val-Asn-Thr-Pro-Glu-His-Val-Val-Pro-Tyr-Gly-Leu-Gly-Ser-Pro-Arg-Ser [Disulfide Bridges: 1-15; 3-11]; Big Endothelin 2 human is available from Sigma with the structure of Cys-Ser-Cys-Ser-Ser-Trp-Leu-Asp-Lys-Glu-Cys-Val-Tyr-Phe-Cys-His-leu-Asp-Ile-Ile-Trp-Val-Asn-Thr-Pro-Glu-Gln-Thr-Ala-Pro-Tyr-Gly-Leu-Gly-Asn-Pro-Pro [Disulfide Bridges: 1-15; 3-11].

In the drug discovery process of the current invention, an ethnopharmacological plant is extracted with a solvent to provide one or more aliquot of ethnopharmacological plant extract. Optionally, such aliquot can be fractionated into groups of fractionates containing compounds selected from individual components of the ethnopharmacological plant extract, and mixtures of individual components of the ethnopharmacological plant extract. Fractionation can be accomplished preferably using a chromatographic separation means such as thin layer chromatography, flash chromatography, column chromatography, low pressure chromatography, medium pressure chromatography, high pressure chromatography, reverse phase chromatography, and combinations thereof. Optionally, an ethnopharmacological plant-extracted aliquot or fractionate thereof can be synthetically modified. Synthetic modification can be achieved by subjecting the aliquot or fractionate any conditions that lead to chemical structure modification. Synthetic modification can be achieved preferably by oxidation, by reduction, by esterification and/or amidation, by hydrolysis, by alkylation, and by combinations thereof. Oxidation can be achieved by exposure to oxidation conditions such as oxygen or air, a peroxide reagent or other oxidizing agents known in the art, or to enzymatic oxidizing conditions to generate one or more synthetic oxidation products of ethnopharmacological plant extract. Reduction can be achieved by exposure to reducing conditions such as catalytic hydrogenation, a hydride reducing agent such as borohydride, diborane, aluminum hydride, and the like, or to enzymatic reducing conditions to generate one or more synthetic reduction products of ethnopharmacological plant extract. Esterification and/or amidation can be achieved by exposure to an esterification or amidification reagent such as any known in the chemical art including an organic acid anhydride, an organic acid mixed anhydride, an organic acid halide, an activated ester containing a chemical leaving group, a carbodiimide reagent in the presence of an alcohol or amine or carboxylic acid, or to enzymatic esterification reagents such as an esterase, a kinase such as can be used to prepare phosphate ester, and the like to generate one or more synthetic esterified products of ethnopharmacological plant extract or to generate one or more synthetic amide products of ethnopharmacological plant extract. Hydrolysis can be achieved by exposure to hydrolysis conditions such as a hydroxide reagent in aqueous solvent, a hydroxide reagent in mixture of an aqueous solvent and an organic solvent such as methanol, ethanol, tetrahydrofuran, dimethyl sulfoxide, and halogenated solvents in the presence of crown ether catalyst to generate one or more synthetic hydrolysis products of ethnopharmacological plant extract. Alkylation can be achieved by exposure to alkylating reagents such as an active ester such as a methanesulfonate, an alkyl halide, an olefin activated for Michael reaction (which is a standard chemical derivatization process to prepare analogs), and the like, to generate one or more synthetic alkylation products of ethnopharmacological plant extract. Alkylation can include aralkylation such as benzylation and substituted benzylation. Organic groups used in modification of the extracts can be selected from any available organic group containing a functional group suitable for reaction with a component of the extract, and preferably selected from those containing from one to forty carbon atoms and which can be saturated, unsaturated, straight or branched chained, which can contain aromatic and substituted aromatic groups, ether groups, and chemically protected groups such as esters of alcohol and of carboxylic acid groups which can be deprotected after modification of the extract. The chemically modified extract aliquot or fractionate can be further separated such as by chromatographic means into groups selected from individual modified components, and combinations thereof.

In the drug discovery process of the current invention, an ethnopharmacological plant extract aliquot, a fractionate thereof, or a synthetically modified version thereof is subjected to a screening procedure to identify the presence of one or more compounds that exhibit a predetermined or desired level of endothelin antagonist activity relative to a reference compound in an endothelin antagonist fluorescence-based assay. The predetermined or desired level of antagonist activity in the binding assay can be from 0.01 percent to 10,000 percent, preferably from 0.1 percent to 1,000 percent, and more preferably from 1 percent to 1,000 percent of that of a selected reference endothelin antagonist compound in the assay.

A compound of the current invention that exhibits a desired level of binding affinity to a receptor in a receptor-ligand binding assay that is greater than or equal to a reference compound of known binding activity to the receptor can be useful as a pharmaceutical or therapeutic endothelin antagonist agent. Such compound can also be useful as a hit or lead compound which can be isolated and whose chemical structure can be determined to identify chemical functional groups and molecular configurations of chemical functional groups or pharmacophores that participate in the binding of endothelin antagonist compound to the endothelin receptor. Each hit or lead compound can be isolated and purified and its structure can be determined such as by spectroscopic, chemical, and X-ray crystallographic methods to identify the chemical functional groups present and the relative orientation of the functional groups, as well as the preferred and possible molecular configurations available to the molecule. A hit or lead compound can be chemically modified as a pure compound or can be chemically modified in a mixture with other compounds or can prepared as a synthetic compound and chemically modified or can be used as a reference compound for the preparation of analogous compounds to optimize binding activity and antagonist activity to the endothelin receptor.

In another aspect of this invention, the methods of this invention can provide endothelin antagonist active compounds as a ligands having activity in a receptor-ligand binding assay that is greater than or equal to the activity a reference compound of known binding activity to or affinity for an endothelin receptor. An endothelin antagonist active compound can be isolated and the chemical structure of such active compound can be determined to identify chemical functional groups and possible molecular configurations of those chemical functional groups or pharmacophores that participate in the binding of the active compound to the receptor. When more than one active compound is present in a mixture of compounds, optionally each active compound can be isolated and purified. The chemical structure of each active compound can be determined such as by spectroscopic, chemical, and X-ray crystallographic methods to identify the chemical functional groups present and the relative orientation of the functional groups, as well as the preferred and possible molecular configurations available to the molecule. An active compound can be chemically modified as a pure compound or can be chemically modified in a mixture with other compounds or can prepared as a synthetic compound and chemically modified or can be used as a reference compound for the preparation of analogous or homologous compounds or compounds having the same or substantially the same pharmacophore to optimize binding activity of such modified compounds as antagonists that bind to an endothelin receptor.

In another aspect, the invention involves treating hypertension, pulmonary hypertension, renal failure and/or kidney failure. The treatment involves administering a therapeutic amount of endothelin antagonist identified and extracted from one or more plants known to provide antagonists to sarafotoxins in snake venom.

### DETAILED DESCRIPTION OF THE INVENTION

The present invention provides a process for the identification of an antagonist of an endothelin selected from the group consisting of endothelin-1, endothelin-2, endothelin-3 and mixtures thereof, said process comprising extraction of ethnopharmacological plants with a solvent followed by evaporation of the solvent to form an aliquot containing at least one component of the extract, optionally purifying said component by chromatographic means, and subjecting said aliquot or said component to a competitive fluorescent binding assay comprising biotinylated endothelin-1, wherein said plants having activity against the effects of one or more sarafotoxin present in snake venom.

The present invention provides a method of detecting the presence of an antagonist of endothelin in an aliquot of an extract of an ethnopharmacological plant, the method comprising
(h) admixing said aliquot and a volume of a solution containing a control amount of biotinylated endothelin-1 into a diagnostically effective plurality of receptors for endothelin-1, said receptors fixed to a plate or substrate;
(i) incubating the admixture in (a) for a time and at a temperature sufficient to allow competitive binding of said antagonist and said biotinylated endothelin-1 to said fixed receptors to occur to form a combination of receptor-bound-antagonist and receptor-bound-biotinylated endothelin-1 as bound receptors;
(j) washing said bound receptors in (b) with a fluorescent-dye-free buffer solution to remove unbound biotinylated endothelin-1;
(k) adding an excess of a solution of streptavidin-Alexa-568 fluorescent dye conjugate to the washed bound receptors in (c);
(l) incubating the streptavidin-Alexa-568 fluorescent dye conjugate with the washed bound receptors of step (c) for a time and at a temperature sufficient to allow binding between biotin and streptavidin to occur to form fluorescent-dye-streptavidin-biotin-endothelin-1-bound receptors in the presence of non-Alexa containing antagonist-bound-receptors;
(m) washing the receptors in (e) with a fluorescent-dye-free buffer solution; and
(n) determining the level of fluorescence emitted from the washed receptors in (f) relative to the level of a fluorescent control,
   wherein said control comprises a comparable plurality of receptors treated as in (a) to (f) but in the absence of the aliquot of said extract, and
   wherein said extract has an activity against the effects of one or more sarafotoxin present in snake venom.

In one aspect, the present invention relates to a method for preparing an isolated extract of an ethnopharmacologic plant. The method includes the steps of extracting one or more part of a plant selected from the group consisting of a root, a portion of a root, a leaf, a bark, a stem, a trunk, a sap, and combinations thereof with an organic solvent to form an extract; concentrating the extract to form a concentrate; washing the concentrate with a water insoluble solvent; and removing the solvent from the concentrate to yield the isolated ethnopharmacologic plant extract. The age of the plants that provide useful quantities of endothelin antagonists can be from the group consisting of seedlings, saplings, mature plants, plants containing one month of growth above ground, plants containing 6 to 12 months of growth such as 10 months of growth above ground, plants that have lived for from one year to two years, plants that have lived from two years to ten years, plants that have lived for ten years to about 100 years, and combinations thereof. The resulting extract can contain at least one endothelin antagonist. The extract can be used in combination with a pharmaceutically acceptable carrier to provide a pharmaceutical composition suitable for use in treatment of a disease in which endothelin contributes to deleterious effects in a patient. In one embodiment, the isolated extract can be used in the treatment of hypertension and coronary heart disease.

Representative ethnopharmacological plant species that are useful in this invention as sources of extract materials, which extracts can contain compounds that exhibit endothelin receptor antagonist activity can be selected from the group consisting of Acacia farnesiana, Acacia sinuata, Achyranthes aspera, Ageratum conyzoides, Alangium salvifolium, Allium cepa, Amaranthus spinosus, Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha, Oroxylum indicum, Oxalis corniculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, Ziziphus oenoplia, Amorphophallus paeoniifolius, Cyamopsis tetragonoloba, Coccinia indica, Physalis minima, Calotropis gigentia, Trichopus zeylanicus, and combinations thereof. Compositions of aliquots of extracts of these plants can contain one or more compounds that are antagonists of endothelin. Aliquots of extracts from two or more plants can be combined and fractionated to provide additional combinations of compounds as mixtures, which mixtures can contain one or more antagonists of endothelin. Combinations of antagonists prepared according to this method can exhibit antagonist behavior with respect to endothelin receptors selected from the group consisting of a receptor for endothelin-1, a receptor for endothelin-2, a receptor for endothelin-3, and combinations thereof. These individual antagonists and mixtures of antagonists can be isolated by chromatographic methods or optionally chemically modified and isolated to provide antagonists of one or more endothelin, for example, an endothelin selected from the group consisting of endothelin-1, endothelin-2, endothelin-3, and combinations thereof.

Extracts obtained according to this invention can be subjected to immediate assay for endothelin receptor antagonist activity by the methods of this invention. Individual components of the extract materials can be purified and isolated as pure compounds that exhibit endothelin receptor antagonist activity. Alternatively, mixtures of compounds can be isolated from the extract materials, wherein at least two components of the mixture exhibit endothelin receptor antagonist activity. Alternatively, a mixture of a compound that exhibits endothelin receptor antagonist activity together with compounds that do not exhibit endothelin receptor antagonist activity can be isolated from the extract material. Crude extract materials can be assayed or screened for endothelin receptor antagonist activity.

Alternatively, extract material obtained according to this invention can be oxidized before it is subjected to the assay of this invention to screen for endothelin receptor antagonist activity. Oxidation can be accomplished by exposing the extract material to oxidizing conditions. Representative oxidizing conditions include exposure of the extract material to oxygen gas particularly when the extract material is dissolved in a solvent or suspended in a solvent; by exposure of the extract material to oxygen in air particularly when the extract material is dissolved in a solvent or suspended in a solvent; by exposure of the extract material to hydrogen peroxide in water or a mixture of water and a compatible organic solvent such as methanol or ethanol or by phase transfer oxidation conditions known in the art; by exposure of the extract material to organic peracidics such as peracetic acid and perphthalic acid particularly when the extract material is dissolved in a solvent such as methylene chloride or suspended in a solvent such as water; by exposure of the extract material to inorganic peracids or inorganic peracid salts such as sodium persulfate, sodium perborate, sodium perchlorate, particularly when the extract material is dissolved in a solvent or suspended in a solvent such as water or a combination of alcohol and water; and by exposure to singlet oxygen generated by sensitized irradiation, particularly when the extract material is dissolved in a solvent or suspended in a solvent. Irradiation useful for singlet oxygen generation from triplet oxygen in the presence extract material, optionally dissolved in a solvent such as methylene chloride, can be that emitted from ultraviolet and/or from visible light sources or from incandescent light sources. In one aspect, one or more components of the extract material can act as a sensitizing agent for singlet oxygen generation in the presence of light. Alternatively, a known singlet oxygen-sensitizing agent such as a benzophenone can be added to the extract or to a solution or suspension of the extract material in the presence of oxygen and irradiation to generate singlet oxygen. Extract materials that are oxidized by exposure to oxidizing conditions can contain additional chemical functional groups such as epoxide groups, alcohol groups, diol groups, vicinal cis-diol groups, vicinal trans diol groups, allylic alcohol groups, carboxylic acid groups, aldehyde groups, and other functional groups such as acetate or other ester groups that are not originally present in the extract materials isolated from natural sources. Additional oxidizing conditions such as treatment with halogens, halogen oxides, nitric oxides, nitrate esters, and acetyl nitrate can introduce additional functional groups into the extract materials.

The process of this invention comprises an extraction of an ethnopharmacological plant. The process can further comprise at least one chemical modification step performed on an aliquot of the extract or on an isolated component of the extract or a mixture thereof, wherein the chemical modification step is selected from the group consisting of oxidation, reduction, esterification, amidation, hydrolysis, and alkylation, and combinations thereof.

Extracts of an ethnopharmacologic plant and components of such extracts of this invention can be obtained from a single plant or a mixture of plants. Extracts can be obtained from the group consisting of an entire ethnopharmacologic plant, a root thereof, a bark thereof, a stem thereof, a leaf thereof, a sap thereof, a branch thereof, a fruit thereof, a flower thereof, a trunk thereof, and combinations thereof.

A plant or plant part such as a root is pulverized into a powder and is extracted with an organic solvent. Useful solvent classes include but are not limited to ether, alkane, aromatic, ester, aralkane, ketone, halogenated alkane, sulfoxide, amide, nitrile, alcohol, supercritical fluid, liquefied petroleum, and combinations thereof. Useful solvents include, for example, a solvent selected from the group consisting of diethyl ether, petroleum ether, hexane, toluene, acetone, acetonitrile, tetrahydrofuran, ethyl acetate, methylene chloride, chloroform, isopropanol, supercritical carbon dioxide, supercritical dimethly ether, liquefied propane, and combinations thereof. The solvent can be removed by evaporation using heat and pressure change conditions to concentrate the extract. Optionally, a solution of the extract in a water insoluble solvent can be washed or extracted with a basic solution such as saturated sodium carbonate, saturated sodium bicarbonate, or a solution containing sodium or potassium hydroxide at pH 8 to 14. Thereafter, the water insoluble solvent can be dried using sodium sulfate or magnesium sulfate, filtered, and the solvent evaporated. The extract can be chromatographed to obtain individual fractions that can be evaluated for endothelin antagonist activity.

An endothelin antagonist according to this invention above can be formulated for administration in a pharmaceutical carrier in accordance with known techniques, for example, those described in Remington, The Science And Practice of Pharmacy (9th Ed. 1995) that is incorporated herein by reference in its entirety.

In the preparation of a pharmaceutical formulation according to the invention, an extracted component or mixture of components which can include one or more physiologically acceptable salts thereof is typically admixed with, inter alia, a pharmaceutically acceptable carrier. The carrier may be a solid or a liquid, or both, and is preferably formulated with the compound as a unit-dose formulation, for example, a tablet, which may contain from 0.01 or 0.5 percent to 95 percent or 99 percent by weight of the extracted component or mixture of components.

The method of administration of a formulation of this invention can be selected from the group consisting of oral, rectal, topical, buccal, sub-lingual, vaginal, parenteral, subcutaneous, intramuscular, intradermal, intravenous, topical, transdermal, transmucosal, inhalation, and combinations thereof. The most suitable route in any given case will depend on the nature and severity of the condition being treated.

The compounds of this invention can be formulated in pharmaceutically acceptable dosage forms such as for injectable use, for oral use, for inhalation use, for transdermal use, for transmembrane use, and the like. Formulations suitable for oral administration may be presented in discrete units or dosage forms, such as capsules, cachets, lozenges, tablets, pills, powders, granules, chewing gum, suspensions, solutions, and the like. Each dosage form contains a predetermined amount of the isolated extraction endothelin antagonist of this invention. Solutions and suspensions can be in an aqueous or non-aqueous liquid or as an oil-in-water or water-in-oil emulsion.

Formulations of the endothelin antagonists of this invention may be prepared by any suitable method of pharmacy. A preferred method comprises the step of bringing into association, for example by mixing, by dissolution, by suspension, by blending, by granulation, and the like an extract or component of an extract of an ethnopharmacologic plant, optionally and sometimes preferably as a component of the extract in purified form, and a pharmaceutically acceptable carrier such as a liquid, for example a liquid selected from the group consisting of water, an aqueous solution of a pharmaceutically acceptable alcohol, a pharmaceutically acceptable oil such as an edible oil such as a triglyceride or mixture of triglycerides of natural sources such as an edible plant oil, an emulsion of a pharmaceutically acceptable oil in an aqueous medium comprising water, and which aqueous medium may contain one or more pharmaceutically acceptable excipients such as an excipient selected from the group consisting of a pH buffering agent, a matrix forming sugar, a pharmaceutically acceptable polymer, a pharmaceutically acceptable tonicity modifying agent, a surface modifier or surfactant useful to form micelles or to form liposomes or to form emulsions. The extract or component can also be combined in solid form with pharmaceutically acceptable excipients such as ingredients used in tablet formation such as release agents and compressing agents, silica, cellulose, methyl cellulose, hydroxypropylcellulose, polyvinylpyrolidinone, gelatin, acacia, magnesium stearate, sodium lauryl sulfate, mannitol, lactose, colorants, dyes, and formed into a dosage form such as a tablet, capsule, caplet, pill, powder, granule, and the like. Optionally, the tablet or related dosage form can be coated with a polymer coating such as an enteric and/or moisture barrier polymer coating such as can be applied by spraying, spray drying, or fluid bed methods.

The extract or component can be combined in an aqueous or aqueous-organic, or an organic liquid solvent together with one or more pharmaceutically acceptable excipient and then dried, for example by spray drying, lyophilization, fluid bed drying, or evaporation to form a solid in which the component or extract is imbibed or uniformly dispersed or suspended. The formulations of the invention can be prepared by admixing, preferably by uniformly and intimately admixing, an extract or component of an extract of an ethnopharmacologic plant, optionally and sometimes preferably in purified form, with a liquid or with a finely divided solid carrier or matrix-forming excipient or mixture of excipients, then, if necessary, shaping the resulting mixture into a dosage form. For example, a tablet may be prepared by compressing or molding a powder or granules containing an isolated extract of this invention, optionally with one or more accessory ingredients. Compressed tablets may be prepared by compressing in a tablet press a mixture of an extract of this invention or component thereof together with one or more pharmaceutically acceptable excipitent materials, which mixture can be in a free-flowing form such as a powder or granules optionally mixed with a pharmaceutically acceptable material selected from the group consisting of a binder, a lubricant, an inert diluent, a surface active agent, a dispersing agent, and combinations thereof. Molded tablets may be made by molding, in a tablet mold machine, a solid powdered mixture of an extract or component of an extract of this invention together with one or more pharmaceutically acceptable excipient, which mixture is moistened with an inert liquid binder such as water or alcohol.

A formulation suitable for buccal or sub-lingual administration to a patient in need of treatment by an antagonist of endothelin of this invention includes a lozenge such as a lozenge comprising an isolated extract or purified component thereof of this invention in a flavored base such as sucrose, acacia, tragacanth, and the like; and a pastille comprising an extract of this invention or a component thereof in an inert base such as gelatin, glycerin, sucrose, acacia, and the like.

The concentration of the antagonist in a dosage form containing an antagonist of this invention depends on the activity and oral bioavailability of the antagonist, and it is at least a therapeutically effective amount of antagonist, preferably from 0.01% by weight to about 50% by weight of the dosage form, more preferably from 0.1% to 40% by weight. Additional concentrations can be selected from the group consisting of 0.1% to 5% by weight, 0.1% to 10% by weight, 0.1% to 20% by weight, 1% to 10% by weight, and 1% to 15% by weight of the dosage form. Depending on the dosage form, pharmaceutically acceptable excipients make up the remainder of the dosage form weight. Excipients such as sugars (lactose, mannitol, sucrose, and the like; polymers such as polyvinylpyrrolidone, poly(vinyl alcohol), pharmaceutically acceptable cellulose derivatives, silica, are useful in solid oral dosage forms.

A formulation of the present invention that is suitable for parenteral administration can comprise a sterile aqueous solution, and a non-aqueous solution in an organic solvent safe for injection of the isolated extracted antagonist of this invention. Useful injectable dosage forms containing an antagonist of this invention preferably are isotonic with the blood of the intended recipient. Tonicity of the dosage form can be adjusted and/or maintained by addition of pharmaceutically acceptable for injection water-soluble excipients such as sugars, buffer salts, and combinations thereof. These dosage forms may optionally contain antioxidants, buffers, bacteriostats, and dissolved solutes that render the formulation isotonic with the blood of the intended recipient. Aqueous and non-aqueous sterile suspensions may include pharmaceutically acceptable suspending agents and thickening agents. Formulations of this invention can be presented in unit-dose or multi-dose containers. For example, for injectable use, a formulation can be sealed in an ampoule or vial, preferably sealed in oxygen-free form such as in a vial under an inert oxygen-free gas such as nitrogen or argon or a mixture thereof. In another embodiment, a dosage form of this invention may be stored in a freeze-dried or lyophilized form containing a small quantity of water, for example from 0.01% to about 5% by weight of the dried dosage form, which dosage form then requires only the addition of a sterile liquid carrier, for example, isotonic aqueous saline solution, and optionally buffered to between about pH 5 to pH 9, or by addition of water-for-injection immediately prior to use. Extemporaneous injection solutions and suspensions may be prepared from sterile powders, granules and tablets of the kind previously described.

A formulation of this invention containing an endothelin antagonist and which is suitable for rectal administration is preferably presented as a unit dose suppository. A suppository dosage form containing an antagonist of this invention may be prepared by admixing an isolated extract of this invention with one or more conventional pharmaceutically acceptable solid carriers, for example, such as cocoa butter, to form a mixture containing the antagonist, and then shaping the resulting mixture.

A formulation of this invention suitable for topical application to skin preferably can be in the form of an ointment, a cream, a lotion, paste, gel, spray, aerosol, oil, or a combination thereof. A pharmaceutically acceptable carrier in this embodiment can be selected from the group consisting of petroleum jelly, lanoline, a polyethylene glycol, a polyethylene glycol ether or ester, an alcohol, a transdermal penetration enhancer, and combinations thereof.

A formulation of this invention suitable for transdermal administration of an endothelin anatgoist of this invention may be presented as a discrete patch dosage form. The patch can be adapted to remain in intimate contact with the epidermis or stratus corneum of a recipient for a prolonged period of time such as from 8 hours to about 48 hours or longer. A formulation suitable for transdermal administration can also be delivered by an iontophoretic delivery mechanism such as be using an applied voltage difference between two portions of the dosage form, each of which is in contact with the skin of a patient.

A therapeutically effective dosage of any antagonist of this invention isolated from an extract of a plant, the use of which is in the scope of present invention, will vary from antagonist compound to antagonist compound, and from patient to patient, and will depend upon factors such as the age of the patient and the diagnosed condition of the patient and the route of delivery of the dosage form to the patient. A therapeutical effective dose and frequency of administration of a dosage form can be determined in accordance with routine pharmacological procedures known to those skilled in the art. Dosage amounts and frequency of administration can vary or change as a function of time and particular condition being treated. For example, a dosage of from about 5 to 40 mg/kg may be suitable for treatment of coronary heart disease.

In one aspect, a preferred dosage of an antagonist of this invention can be from about 20 to about 35 mg/kg to have therapeutic efficacy.

In one aspect, an antagonist of this invention can be in the form of a salt, such as a protonated amine form or a deprotonated carboxylate or other acid form. Intravenous dosage forms can sometimes be up to about 20 mg/kg of antagonist. A preferred dosage from about 30 mg/kg to about 50 mg/kg may be employed for oral administration. A preferred dosage from about 20 mg/kg to 30 mg/kg may be employed for intramuscular injection. The frequency of administration of a dosage form of this invention can be once, or twice, or three times, or four times per day. A useful duration of treatment of a patient can be from about one or two days, up to five or seven days, up to two or three weeks, or until symptoms of a disease state in a patient are essentially controlled. An antagonist of this invention isolated by extraction can be used in the treatment of diseased states such as coronary heart disease, antifungal infection, bacterial infection, acne, eczema, and the like. An antagonist of this invention can be used as an anticoagulant, as a cholesterol lowering or control agent, and to protect myocardial tissue from hypoxia-induced contractile failure.

The method of this invention uses a fluorescent-based ligand-receptor interaction assay technique. A biotin-linked-to-endothelin-1 conjugate, herein sometimes referred to as biotinylated endothelin-1 or as BT-ET-1, is used as a competitive-binding ligand for an endothelin receptor in the endothelin-receptor binding assays of this invention that are used to identify antagonists of endothelin extracted from ethnopharmacological plants that have activity in the treatment of humans against the effects of sarafotoxins present in snake venom injected into a human during a snake bite. Biotinylated endothelin-1 binds to a receptor for endothelin on the surface of a cell via the endothelin-1 portion of the conjugate. The receptor-bound biotinylated endothelin-1 is subsequently treated with a streptavidin-coupled-Alexa-568 dye conjugate (available from Molecular Probes, Eugene Oreg.) which binds specifically to the biotin segment of the biotinylated endothelin-1 conjugate. The amount of streptavidin-Alexa-568 dye complex specifically bound to a biotinylated endothelin-1 conjugate bound to an endothelin receptor in the assay is proportional to the amount of biotinylated endothelin-1 bound to the endothelin receptor. The binding of the endothelin portion of the biotinylated endothelin-1 conjugate is competitive with other molecules such as agonists and antagonists of endothelin-1. The binding of the endothelin portion of the biotinylated endothelin-1 conjugate is inhibited in the presence of an endothelin-receptor antagonist. When any non-bound fluorescent conjugates are removed from the site of fluorescence measurement in the assay and when the background or ambient fluorescence level is subtracted from the measured fluorescence value, relative to a maximum fluorescence level determined in a control that uses only biotinylated endothelin-1 as a control, a net decrease in fluorescence level measured in the competitive binding of biotinylated endothelin-1 to the endothelin receptor in the presence of an aliquot of an extract of the ethnopharmacologic plants or in the presence of a purified component of such extract is an indicator of the presence of an endothelin receptor antagonist that binds to the endothelin receptor, which antagonist is thus detected to be present in the extract or aliquot of the extract of the plant.

A relative measure of the level of endothelin receptor antagonist activity in an aliquot of an ethnopharmacologic plant extract of this invention can be determined relative to the activity of a reference endothelin receptor antagonist as a control, optionally for which control an absolute binding constant of the reference antagonist to the receptor is known, and optionally for which control antagonist the chemical structure is known. The relative level of endothelin receptor antagonist activity versus the level of antagonist activity of a known endothelin receptor antagonist can be determined by measuring the net decrease in fluorescence output or the relative net level of fluorescence output in the assay of this invention compared relative to a control. When the fluorescence assay is performed in the presence of a sufficient biotinylated endothelin-1 to bind specifically to all available binding receptor sites, the level of fluorescence measured comprises a maximum fluorescence level, net of background or ambient fluorescence, possible with the assay. This maximum fluorescence level correlates with a saturation of all available endothelin receptors by the endothelin-1 conjugate that is further conjugated to the fluorescent dye. A decrease in net fluorescence correlates to the level of binding caused by any antagonist compounds in a competitive binding assay. Competitive binding of biotinylated endothelin-1-ligand to endothelin-receptor is a useful method for the quantification of endothelin antagonists in a drug discovery program. This assay provides sensitivity comparable to that obtained using endothelin-receptor radioligand binding assays. Specific binding as used herein is defined as the difference between total binding and non-specific binding. IC50 values can be determined after logit/log transformation of binding data obtained from the assay of this invention.

Bosentan is a known, non-selective endothelin-receptor antagonist. It can be used herein as a control or standard for calibration of the assay and to establish a comparative relative level of endothelin-receptor antagonist binding activity exhibited by unknown compounds that are present in the plant extracts of this invention. A lower relative sensitivity limit of antagonist activity is in the range of 20 to 50 ng/mL. Concentrations of antagonist above a relative upper limit of about 2 to 10 mg/mL can be measured after dilution of the aliquot of extract with a buffer to below this limit. A suitable buffer is Tris buffer. Accuracy and precision with respect to detection of competitive antagonist activity can be increased by performing assay measurements on aliquots of extracts in triplicate or in quadruplicate determinations. Endothelin antagonists can be formulated for oral use such as in tablets. An antagonist of this invention can be administered orally to improve the exercise ability of patients with a rare but fatal lung disorder called pulmonary arterial hypertension (PAH). Pulmonary arterial hypertension is defined as abnormally high blood pressure in the arteries between the heart and lungs. PAH significantly reduces the ability of patients to exert themselves physically without becoming short of breath. PAH significantly shortens the life span of patients because it leads to heart failure. An endothelin antagonist of this invention blocks the action of endothelin. Endothelin narrows blood vessels and elevates blood pressure. Although endothelin is present in normal healthy people, high concentrations of the hormone endothelin can be found in the plasma and lungs of patients with PAH.

Reduction in levels of maximum fluorescence caused by the presence of one or more antagonists of endothelin in an aliquot of extract of this invention are from 0.1% to 99.9% of control levels. A useful reduction is in the range of 1% to 99% in some cases, and in the range from 10% to 89% in other cases.

The invention described and claimed herein can be further appreciated by one skilled in the art through reference to the examples which follow. These examples are provided merely to illustrate several aspects of the invention and shall not be construed to limit the invention in any way. Using the Fluorescent-based receptor assay technique of this invention, analysis of extracts samples with unknown concentration of endothelin-receptor antagonistic properties can be completed in less than four to six hours of assay time. In comparison, use of the conventional radioassay methodology requires approximately 24 hours. The assay system allows quantitative analysis of potential selective and nonselective endothelin-receptor antagonists from various sources and can be adapted for high through put screening. In addition, this assay is useful for pharmacokinetic studies in in vitro and animal model studies such as pharmacokinetic data analysis. Pharmacokinetic data is essential for final selection of endothelin (ET) antagonists to be considered for clinical development. Thus, an accurate, sensitive, and fast method to assess plasma levels after both parenteral and oral application is needed. In this method small amounts such as 1 to 10 microliters of plasma can be sufficient for a plasma level assay of antagonist activity and can facilitate repeated sampling in small animals, such as rats.

### EXAMPLES

### Example 1

### Extraction of an Ethnopharmacological Plant

Roots are collected from a bed containing an ethnopharmacologic plant. The roots are washed in tap water, air dried at 45° C. and ground in a Willey mill to provide particles of ground root of about 10 micrometer size. Three 150 g samples of powdered root are each extracted with 1 L of diethyl ether in a soxhlet extractor at reflux for 2 hr. The extract ether extract is washed with 5% sodium carbonate solution, the ether is dried with sodium sulfate, and the solvent is removed on a rotary evaporator to obtain a dried crude extract. The crude extract is subjected to medium pressure flash column chromatography using silica gel 60 (230-400 mesh) in a glass column with a 30 mm diameter and 1000 mm length that is packed with silica gel in hexane slurry. The crude sample (1 g) is loaded on to the column by first adsorbing it on to 1 g of silica gel. The column is eluted with diethyl ether stepwise with increasing portions of acetone in ether, stepwise with increasing portions of methanol in acetone, and finally with pure methanol. The flow rate of the eluent is maintained at about 2 ml/min. All fractions are collected as 10 ml volumes, and each is concentrated to dryness in a savant concentrator. Each fraction is subjected to thin layer chromatography on silica gel to identify components with similar elution times. Fractions containing similar components optionally can be combined, and analyzed for endothelin antagonist activity or fractions can be subjected to preparative thin layer chromatography to separate the fractions or combined similar fractions into components consisting of individual compounds and mixtures of individual compounds having similar rf values. Additional purification can be performed using high pressure liquid chromatography (HPLC) typically with the aid of a gradient-capable solvent delivery system and diode array detector or refractive index detector. A sample of the extract or eluate isolated by thin layer chromatography can be charged on a 100 by 2.1 mm column (5 um Hypersil C18 reversed phase), and can be eluted with a water-acetonitrile gradient with a flow rate of 0.6 ml/min. Individual fractions can be collected, dried, typically under nitrogen or subjected to air oxidation by exposure to air, and optionally freeze-dried to individual component fractions which can be characterized spectroscopically and which are evaluated for endothelin antagonist activity.

### Example 2

The procedure of example 1 is repeated using roots collected from a grove consisting of from two to 100 in number of an ethnopharmacologic plant, which plants are growing within a one hundred square meters of each other.

### Example 3

The procedure of example 1 is repeated using roots collected from at least two individual plants of the same genus of ethnopharmacologic plant that are growing in separate locations.

### Example 4

### Endothelin Receptor Antagonist Assay Using Biotinylated Endothelin-1

This invention comprises a fluorescent-based ligand-receptor interaction assay technique. A biotin-linked-to-endothelin-1 conjugate is used as a competitive-binding ligand for an endothelin receptor in an endothelin-receptor binding assays to identify antagonists of endothelin extracted from ethnopharmacological plants that have activity against the effects of sarafotoxins present in snake venom. Biotinylated endothelin-1 binds to a receptor for endothelin on the surface of a cell via the endothelin-1 portion of the conjugate. The receptor-bound biotinylated endothelin-1 is subsequently treated with a streptavidin-coupled-Alexa-568 dye conjugate (available from Molecular Probes, Eugene Oreg.) which binds specifically to the biotin segment of the biotinylated endothelin-1 conjugate. The amount of streptavidin-Alexa-568 dye complex specifically bound to a biotinylated endothelin-1 conjugate bound to an endothelin receptor in the assay is proportional to the amount of biotinylated endothelin-1 bound to the endothelin receptor. The binding of the endothelin portion of the biotinylated endothelin-1 conjugate is competitive with agonists and antagonists of endothelin-1. The binding of the endothelin portion of the biotinylated endothelin-1 conjugate is inhibited in the presence of an endothelin-receptor antagonist. When non-bound fluorescent conjugates are removed from the site of fluorescence measurement in the assay and when the background ambient fluorescence level is subtracted from the measured fluorescence value, relative to a maximum fluorescence level determined in a control that uses only biotinylated endothelin-1 as a control, a net decrease in fluorescence level measured in the competitive binding of biotinylated endothelin-1 to the endothelin receptor in the presence of an aliquot of an extract of the ethnopharmacologic plant or in the presence of a purified component of such extract is an indicator of the presence of an endothelin receptor antagonist in the extract or aliquot.

A level of endothelin receptor antagonist activity in an aliquot of an ethnopharmacologic plant extract can be determined relative to the activity of a reference endothelin receptor antagonist as a control by measuring the net decrease in fluorescence output or the relative net level of fluorescence output in the assay compared relative to a control. A maximum fluorescence level is obtained when the fluorescence assay is performed in the presence of a sufficient biotinylated endothelin-1 to bind specifically to all available binding receptor sites. This maximum fluorescence level correlates with a saturation of all available endothelin receptors by the endothelin-1 conjugate that is further conjugated to the fluorescent dye. A decrease in net fluorescence correlates to the level of binding caused by any antagonist compounds in a competitive binding assay.

Bosentan, a known, non-selective endothelin-receptor antagonist is used as a control or standard for calibration of the assay and to establish a comparative relative level of endothelin-receptor antagonist binding activity exhibited by an unknown compound present in a plant extracts of example 1. A lower relative sensitivity limit of antagonist activity is in the range of 20 to 50 ng/mL. Concentrations of antagonist above a relative upper limit of about 2 to 10 mg/mL can be measured after dilution with TRIS buffer to below this limit. Accuracy and precision with respect to detection of competitive antagonist activity can be increased by performing assay measurements on aliquots of extracts in triplicate or in quadruplicate determinations.

The following materials are used. Fluoroplates are available from VWR Scientific (for example, see catalog number 13503-314) (Whatmann reference number 7716-2380) and plates pre-coated with 2% gelatin (see VWR catalog number EM-GX0048-1). NIH 3T3 cell line (ATCC number CRL-1658); note that recombinant endothelin_{A} (which is sometimes referred to herein as endothelin-sub-A and as ET_{A}) receptor and recombinant endothelin_{B} (which is sometimes referred to herein as endothelin-sub-B and as ET_{B}) receptor can also be used. Buffers and reagents for preparing NIH 3T3 cells include: fetal calf serum (GibcoBRL catalog number 16170-078); media (ATCC catalog number 30-2002); Pen/Strep solution (Sigma catalog number T4549 otherwise referred to as Trypsin solution from porcine pancreas, 25 g/l porcine trypsin in 0.9% NaCl, cell culture, sterile-filtered); T-75 flasks (VWR catalog number 29186-105); T-225 flasks (VWR catalog number 29560-959); PBS (1X) (GibcoBRL, Catalog number 20012-027); Trypsan (Sigma, Catalog number T4549); BSA or bovine serum albumin (VWR catalog number JT3827-1); FAM-labeled Endothelin-1 (Phoenix Pharmaceuticals catalog number FG-023-01) which is useful at 0.01 nmole/mL, at 0.001 nmole/mL, and at 0.0001 nmole/mL where FAM is a carboxyfluorescein selected from the group consisting of 5-carboxyfluorescein, 6-Carboxyfluorescein, 5(6)-Carboxyfluorescein, and combinations thereof; streptavidin Alexa-568 (Molecular Probes catalog number S-11226) where a stock solution is prepared by dissolving 1 mg of streptavidin Alexa-568 powder in 10 mL of PBS (1×), which stock solution can be further diluted to useful concentrations at dilutions of 1:50 as well as in the range of 1:100 to 1:1600; biotinylated endothelin-1 (Peninsula laboratories Europe LTD (Bachem) catalog number 6937) dissolved in 0.01 M ammonium bicarbonate solution. Plate reader: FL600 Microplate Fluorescence reader (Bio-Tek Instruments part#6001000) using KC4 software used with filters Excitation 485/20 and Emission 530/25 for the FAM-labeled Endothelin-1, and with filters Excitation 590\20 and Emission 590\35 for the biotinylated endothelin-1. Controls used for the assay are known endothelin antagonists such as BQ-610 (Sigma Catalog number B151) with a structure of N-[1-Formyl-N-[N-[(hexahydro-1H-azepin-1-yl)carbonyl]-L-leucyl]-D-tryptophyl]-D-tryptophan); PD151242 (Sigma catalog number P208) with a structure of N-[N-[N-[(hexahydro-1H-azepin-1-yl)carbonyl]-L-leucyl]-1-methyl-D-tryptophyl]-D-tyrosine; and BQ-788 (Sigma catalog number B 157) with a structure of 2,6-dimethylpiperidinecarbonyl-g-methyl-Leu-Nin-(methoxycarbonyl)-D-Trp-D-Nle-N-[N-[N-[(2,6-dimethyl-1-piperidinyl)carbonyl]-4-methyl-L-leucyl]-1-(methoxycarbonyl)-D-tryptophyl]-D-norleucine, sodium salt.

For fluorescence controls, fluorescent compound(s) are added to wells with or without cells. The cells are not washed out in order to determine if there is any background fluorescence in the fluorescent solutions. For background controls, fluorescence can be measured in wells with cells and media only. For 3T3 cell line incubation, the cells are not allowed to become more that 70% confluent when expanding in flasks. Cells are seeded into 96-well plates at 2,500 cells per well, at 5,000 cells per well, and at 10,0000 cells per well. A suitable range is 2,500 to 10,000 cells per well.

Cells from a T-225 or T-75 flask are trypsinized, spun down and resuspended in a volume of buffer sufficient to permit seeding of a gelatinized fluoroplate with 2.5, 5, or 10,000 cells per well. The fluoroplate is incubated for 24 hours whereupon test substances are added and controls in IMDM [IMDM is Iscove's Modified Dulbecco's Medium used for cell culture and available from Sigma-Aldrich as product 13390]. The plates are then incubated for 1.5 hours, each well is aspirated, biotinylated endothelin-1 is added, and the plates are incubated for another 1.5 hours. The biotinylated endothelin-1 is than aspirated and the wells are washed twice with PBS. Streptavidin Alexa-568 conjugate is than added and the wells are incubated for another 1 hour. The wells are aspirated and washed twice with PBS; the last wash is left in the wells. A fluorescent reading is taken with a FL600 Microplate Fluorescence reader.

### Example 5

### Endothelin Antagonist Assay Using FAM-labeled Endothelin-1

Cells from a T-225 or T-75 flask are tripsinized, spun down, and resuspended in volume appropriate to seed a gelatinized fluoroplate with 2.5, 5 or 10,000 cells per well. The cells are incubated for 24 hours, test substances are added and controls in IMDM, and the plates are incubated for another 1.5 hours. Each well is aspirated and FAM-labeled Endothelin-1 is added diluted in IMDM (without phenol red indicator) supplemented with 10% FBS, and incubated for another 1.5 hours. Each non-control well is aspirated and wash twice with 100 uL of IMDM (without phenol red indicator) supplemented with 10% FBS. The last wash is left in the wells and a fluorescent reading is taken with a FL600 Microplate Fluorescence reader.

### Example 6

(a) Preparation of Fluoroplates for Assay of Extracts for Endothelin Antagonist Activity.

Fluoroplates such as those containing 6, 24, 48, or 96 wells are precoated with polyethylene imine (0.1%) in 0.2 M Na2HPO4/citrate phosphate buffer at pH 7.4 for 24 hours at 4° C. The plates are then washed three times with 0.2 M Na2HPO4/citrate phosphate buffer at pH 7.4. The wells are then seeded with cultured fibroblasts (NIH-T3T cells) that contain endothelin receptor. Nonspecific binding sites are blocked by treatment of the wells with 1% BSA in 1× PBS for 1 hour at room temperature.

(b) Competitive Binding Assay of Extracts for Endothelin Antagonist Activity

To each well of the fluoroplate is added 100 mL of Tris buffer consisting of 50 mM Tris, 1 mM EDTA, 0.01% NaN3, 0.5% BSA, 25 mM MnCl2 at pH 7.4. To each well is then added 10 to 50 mL of a sample aliquot of plant extract or purified component of a plant extract and 100 mL of Tris buffer containing BT-ET-1 at a concentration of 1 to 10 mM. The plate is then incubated for 90 min at 37° C. Alternatively, the plate can be incubated for three hours at room temperature. The wells are then washed twice with phosphate buffer. To each well in the assay is added Streptavidin Alexa-568 solution at a concentration of about 10 mM. The plate is then incubated for 1 hour at room temperature, and then washed twice with phosphate buffer. Fluorescence output levels are then measured using a fluorescent microplate reader with excitation at 573 nm and emission at 596 nm.

### Example 7

The procedure of Example 6 is repeated using recombinant ETA receptor at 5 mg protein per well in 100 mL phosphate buffer for 16 to 24 hours instead of NIH-T3T cells in the fluoroplates. Optionally, these plates can be stored and then used in the assay for about six weeks after seeding with recombinant ETA receptor without significant loss of endothelin binding capacity.

### Example 8

The procedure of Example 6 is repeated using recombinant ETB receptor at 5 mg protein per well in 100 mL phosphate buffer for 16 to 24 hours instead of NIH-T3T cells in the fluoroplates. Optionally, these plates can be stored and then used in the assay for about six weeks after seeding with recombinant ETB receptor without significant loss of endothelin binding capacity.

1. A process for the identification of an antagonist of an endothelin selected from the group consisting of endothelin-1, endothelin-2, endothelin-3 and mixtures thereof, said process comprising extraction of ethnopharmacological plants known to be effective against at least one sarafotoxin in snake venom with a solvent followed by evaporation of the solvent to form an aliquot containing at least one component of the extract, purifying said component, and subjecting said aliquot or said component to a competitive fluorescent binding assay comprising biotinylated endothelin-1.

2. The process of claim 1, wherein the plant is selected from the group consisting of Acacia farnesiana, Acacia sinuata, Achyranthes aspera, Ageratum conyzoides, Alangium salvifolium, Allium cepa, Amaranthus spinosus, Amorphophallus paeoniifolius, Anthocephalus chinensis, Ardisia solanaceae, Artocarpus integrifolia, Asclepias curasavica, Asparagus racemosus, Atalantia monophylla, Baliospermum montanum, Bauhinia pupurea, Bauhinia tomentosa, Bauhinia variegata, Bidens bipinnata, Bixa orellana, Boerhaavia diffusa, Bombax ceiba, Boswellia serrata, Buchanania lanzan, Bulbostylis barbata, Calotropis gigantea, Capparis zeylanica, Careya arborea, Cassia fistula, Cassia occidentalis, Cassia tora, Cassine glauca, Cedrus deodara, Chomaesyce hirta, Chomaesyce prostrata, Cissampelas pareira, Cissus pallida, Cissus quadrangularis, Clerodendrum serratum, Coccinia indica, Conyza canadensis, Cordia myxa, Coriandrum sativum, Crataeva religiosa, Croton sparsiflorous, Cryptolepis buchanani, Curculigo orchioides, Cyamopsis tetragonoloba, Cyperus rotundus, Datura innoxia, Datura metel, Dolichandrone crispa, Embelia ribes, Erythrina indica, Erythrina stricta, Eupatorium odoratum, Ficus benghalensis, Ficus religiosa, Gardenia latifolia, Glycosmis arborea, Gmelina arborea, Grangea sp., Gymnema sylvestre, Hemidesmus indicus, Heteropogon contortus, Ichnocarpus frutescens, Indoneesiella echiodes, Ipomoea hederifolia, Kalanchoe pinnata, Lannea coromandalica, Leucas aspera, Luffa acutangula, Madhuca indica, Mallotus phillipensis, Melochia corchorifolia, Melothria sp., Mesua nagassarium, Mimosa pudica, Moringa oleifera, Mucuna pruriens, Nerium indicum, Nyctanthes arbor-tristis, Ocimum americanum, Ocimum tenuiflorum, Opuntia monocantha, Oroxylum indicum, Oxalis comiculata, Pandanus fascicularis, Pergularia daemia, Phyllanthus acidus, Physalis minima, Piper longum, Plantago ovata, Polycarpea corymbosa, Polygala erioptera, Polygonum barbatum, Pongamia glabra, Rhus succedanea, Sapindus laurifolius, Sarcostemma acidum, Sida acuta, Smilax zeylanica, Solanum torvum, Solanum trilobatum, Strychnos nux-vomica, Tamarindus indica, Tephrosia purpurea, Tephrosia tinctoria, Terminalia bellirica, Thottea siliquosa, Tinosporia cardifolia, Tragia connabina, Tragia involucrata, Trichopus zeylanicus, Vetiveria zizaniodes, Vitex altissima, Wattakaka volubilis, Xanthium indicum, and Ziziphus oenoplia. Additional species include:
Amorphophallus paeoniifolius, Cyamopsis tetragonoloba, Coccinia indica, Physalis minima, Calotropis gigentia, Trichopus zeylanicus, and mixtures thereof.

3. The process of claim 1 further comprising at least one chemical modification step.

4. The process of claim 2 further comprising at least one chemical modification step.

5. The process of claim 3, wherein the chemical modification step is selected from the group consisting of oxidation, reduction, esterification, amidation, hydrolysis, and alkylation, and combinations thereof.

6. The process of claim 4, wherein the chemical modification step is selected from the group consisting of oxidation, reduction, esterification, amidation, hydrolysis, and alkylation, and combinations thereof.

7. An antagonist of endothelin-1 prepared by the method of claim 1.

8. An antagonist of endothelin-2 prepared by the method of claim 1.

9. An antagonist of endothelin-3 prepared by the method of claim 1.

10. A method of detecting the presence of an antagonist of endothelin in an aliquot of an extract of an ethnopharmacological plant, the method comprising
(o) admixing said aliquot and a volume of a solution containing a control amount of biotinylated endothelin-1 into a diagnostically effective plurality of receptors for endothelin-1, said receptors fixed to a plate or substrate;
(p) incubating the admixture in (a) for a time and at a temperature sufficient to allow competitive binding of said antagonist and said biotinylated endothelin-1 to said fixed receptors to occur to form a combination of receptor-bound-antagonist and receptor-bound-biotinylated endothelin-1 as bound receptors;
(q) washing said bound receptors in (b) with a fluorescent-dye-free buffer solution to remove unbound biotinylated endothelin-1;
(r) adding an excess of a solution of streptavidin-Alexa-568 fluorescent dye conjugate to the washed bound receptors in (c);
(s) incubating the streptavidin-Alexa-568 fluorescent dye conjugate with the washed bound receptors of step (c) for a time and at a temperature sufficient to allow binding between biotin and streptavidin to occur to form fluorescent-dye-streptavidin-biotin-endothelin-1-bound receptors in the presence of non-Alexa containing antagonist-bound-receptors;
(t) washing the receptors in (e) with a fluorescent-dye-free buffer solution; and
(u) determining the level of fluorescence emitted from the washed receptors in (f) relative to the level of a fluorescent control,
wherein said control comprises a comparable plurality of receptors treated as in (a) to (f) but in the absence of the aliquot of said extract, and
wherein said extract has an activity against the effects of one or more sarafotoxin present in snake venom.

11. A method of treating endothelin caused conditions in a patient, comprising administering a therapeutically effective account of endothelin antagonist to the patient, said endothelin antagonist having been obtained from extraction of ethnopharmological plants known to be effective against at least one sarafotoxin in snake venom.

12. The method of claim 11, wherein said condition is hypertension.

13. The method of claim 11, wherein said condition is pulmonary hypertension.

14. The method of claim 11, wherein said condition is renal failure.

15. The method of claim 11, wherein said condition is kidney failure.

16. The method of claim 11, wherein said administering is done by one selected from the group consisting of oral, rectal, topical, buccal, sub-liquid, vaginal, parenteral, subcutaneous, intramuscular, intradermal, intravenous, topical, transdermal, transmucusol, inhalation and combinations thereof.

17. The method of claim 11, wherein said endothelin antagonist is prepared as a formulation for administering mixed with a pharmaceutically acceptable carrier.

18. The method of claim 17, wherein the formulation further comprises at least one physiologically acceptable salt in a liquid.

19. The method of claim 17, wherein the formulation further comprises at least one physiologically acceptable salt in a tablet.
